(19) United States
(12) Patent Application Publication
(10) Pub. No.: US 2008/0292733 A1
(43) Pub. Date: Nov. 27, 2008

(54) Antioxidant and Fe2+ Chelating Properties of Herbal Extracts

(76) Inventors: Sharla K. Sutherland, Edmonton (CA); Jacqueline J. Shan, Edmonton (CA)

Correspondence Address: ROTHWELL, FIGG, ERNST & MANBECK, P.C., 1425 K STREET, N.W., SUITE 800, WASHINGTON, DC, 20005, US

(21) Appl. No.: 12/090,957

(22) PCT Filed: Oct. 16, 2006
(86) PCT No.: PCT/IB06/02880

§ 371 Date: Aug. 11, 2008

Related U.S. Application Data
(60) Provisional application No. 60/728,747, filed on Oct. 21, 2005.

(51) Int. Cl. A61K 36/38; A61K 31/56; A61P 35/00; A61P 9/00; A61P 31/00; A61P 1/00; A61P 25/00; A61K 36/16

(52) U.S. Cl. 424/730; 514/169; 424/752

(57) ABSTRACT

The present invention is directed to antioxidative and antiproliferative agents containing North American ginseng (proprietary extract HT1001) alone or in combination with Ginkgo biloba (GB), Saint John's Wort (SJW), ginkgolides, flavonoids, ginsenosides, hypericin, and/or hyperforin. The antioxidative and antiproliferative agents according to the present invention show significant inhibition of Fe²⁺-catalyzed lipid peroxidation and Fe²⁺ chelation activity, the ability to scavenge hydroxyl free radicals, the ability to reduce neuroblastoma cell numbers, and/or the ability to promote neurite outgrowth. These effects are consistent with the behavior of Fe²⁺ chelators and the variance in both type and magnitude of effects exerted by the chemical components suggests a synergistic mechanism of action.

### BACKGROUND OF THE INVENTION

For many centuries, Chinese herbal medicine has utilized ginseng, Ginkgo biloba (GB), and Saint John's Wort (Hypericum perforum, SJW) for managing a wide variety of neurological conditions and ailments. A recent meta-analysis has described beneficial effects of GB extracts on cognition, mood, emotional function and quality of life for treatment of dementia with no significant adverse effects, although the need for larger clinical trials was stressed [Birks J, Grimley E V, van D M. Ginkgo biloba for cognitive impairment and dementia. Cochrane Database Syst Rev 2002; (4):CD003120]. Similarly, North American ginseng (Panax quinquefolius) extracts have also been reported to improve memory [Sloley B D, Pang P K, Huang B H, Ba F, Li F L, Benishin C G, Greenshaw A J, Shan J J. American ginseng extract reduces scopolamine-induced amnesia in a spatial learning task. J Psychiatry Neurosci 1999 November; 24(5):442-52] and its component ginsenosides Rb1 and Rg1 to exert neuroprotective effects [Rudakewich M, Ba F, Benishin C G. Neurotrophic and neuroprotective actions of ginsenosides Rb(1) and Rg(1). Planta Med 2001 August; 67(6):533-7]. The beneficial attributes of ginseng are attributed to its saponin content, a mixture of dammarane triterpene glucosides referred to collectively as ginsenosides. Some ginsenosides have been isolated, and their structure determined. Such ginsenosides include Rb1, Rb2, Rc, Rd, Re, Rf and Rg (see U.S. Pat. No. 4,157,894 to Bombardelli). SJW, approved in Germany for the treatment of mild to moderate depression, was shown in a recent meta-analysis to offer significantly superior results to synthetic antidepressants with a more favorable side effects profile [Roder C, Schaefer M, Leucht S., Meta-analysis of effectiveness and tolerability of treatment of mild to moderate depression with St. John's Wort, Fortschr Neurol Psychiatr 2004 July; 72(6):330-43].

Oxidative stress has been implicated in various neurological pathologies therefore some of the effects of GB, North American ginseng, and SJW extracts may be attributed to their inherent antioxidant activities. Flavonoids, which are known to be major bioactive components contained in GB and SJW, have been shown to possess antioxidant activity including prevention of lipid peroxidation, free radical scavenging, and Fe²⁺ chelation [Rice-Evans C A, Miller N J, Paganga G. Structure-antioxidant activity relationships of flavonoids and phenolic acids. Free Radic Biol Med 1996; 20(7):933-56; Sloley B D, Urichuk L J, Morley P, Durkin J, Shan J J, Pang P K, Coutts R T. Identification of kaemferol as a monoamine oxidase inhibitor and potential Neuroprotectant in extracts of Ginkgo biloba leaves. J Pharm Pharmacol 2000 May; 52(4):451-9; Sloley B D, Urichuk L J, Ling L, Gu L D, Coutts R T, Pang P K, Shan J J. Chemical and pharmacological evaluation of Hypericum perforatum extracts. Acta Pharmacol Sin 2000; 21(12):1145-52].

Antioxidant effects have also been described for the ginsenosides, Rb1 and Rg3 [Kim Y C, Kim S R, Markelonis G J, Oh T H. Ginsenosides Rb1 and Rg3 protect cultured rat cortical cells from glutamate-induced neurodegeneration. J Neurosci Res 1998 September 15; 53(4):426-32; Lim J H, Wen T C, Matsuda S, Tanaka J, Maeda N, Peng H, Aburaya J, Ishihara K, Sakanaka M. Protection of ischemic hippocampal neurons by ginsenoside Rb1, a main ingredient of ginseng root. Neurosci Res 1997 July; 28(3):191-200] and SJW extracts [Jang M H, Lee T H, Shin M C, Bahn G H, Kim J W, Shin D H, Kim E H, Kim C J. Protective effect of Hypericum perforatum Linn (St. John's wort) against hydrogen peroxide-induced apoptosis on human neuroblastoma cells. Neurosci Lett 2002 September 30; 329(2):177-80]. Extracts which exert their antioxidant cellular protective effects through Fe²⁺ chelation may also be hypothesized to exhibit anti-proliferative/pro-apoptotic activity, depending on the conditions and nature of the model used. Because Fe²⁺ is a critical element for the proliferation of cells, neoplastic cells have an increased Fe²⁺ demand. Anti-cancer activity has also been demonstrated clinically for various pathologies including neuroblastoma Fe²⁺ chelators [Donfrancesco A, Deb G, De S L, Cozza R, Castellano A. Role of deferoxamine in tumor therapy. Acta Haematol 1996; 95(1):66-9]. In addition to their defined anti-proliferative effects [Renton F J, Jeitner T M. Cell cycle-dependent inhibition of the proliferation of human neural tumor cell lines by iron chelators. Biochem Pharmacol 1996 July 14; 51(11):1553-61], Fe²⁺ chelators may also induce apoptosis [Fan L, Iyer J, Zhu S, Frick K K, Wada R K, Eskenazi A E, Berg P E, Ikegaki N, Kennett R H, Frantz C N. Inhibition of N-myc expression and induction of apoptosis by iron chelation in human neuroblastoma cells. Cancer Res 2001 March 1; 61(3): 1073-9] and promote cellular differentiation in several tumor lines including embryonal carcinoma F9 [Tanaka T, Muto N, Ido Y, Itoh N, Tanaka K. Induction of embryonal carcinoma cell differentiation by deferoxamine, a potent therapeutic iron chelator. Biochim Biophys Acta 1997 July 5; 1357(1):91-7].

Recently, a pilot study of a unique extract combination product (CVT-E033, AD-fX® CV Technologies, Edmonton, Alberta) containing a patented proprietary North American ginseng extract, HT1001, and GB used in the treatment of attention-deficit hyperactivity disorder (ADHD) suggested that it may improve ADHD symptoms, including cognitive problems [Lyon M R, Cline J C, Totosy de Z J, Shan J J, Pang P, Benishin C. Effect of the herbal extract combination Panax quinquefolium and Ginkgo biloba on attention-deficit hyperactivity disorder: a pilot study. J Psychiatry Neurosci 2001 June; 26(3):221-8]. This study, in addition to reports of neuroprotective/neurotropic and antioxidant effects of North American ginseng, GB, and SJW has prompted investigation of the mechanisms of action of these substances and the combinations thereof, CVT-E033 and CVT-E036 (Menta-fX®; GB, North American ginseng, and SJW).

U.S. Pat. Nos. 5,137,878 and 6,083,932 to Pang et al contain a discussion of prior art extracts. The complete disclosure of U.S. Pat. No. 5,137,878 and U.S. Pat. No. 6,083,932 is hereby incorporated by reference in this application. U.S. Pat. No. 5,137,878 discloses that ginsenosides Rb1 and Rg1 enhance the availability of ACh in the cortical and hippocampal regions of the brain and alleviate the symptoms of Alzheimer-type senile dementia. The patent also discloses a process for isolating ginsenoside Rb1. U.S. Pat. No. 6,083,932 discloses pharmaceutical compositions derived from ginseng and methods for the treatment of a variety of brain conditions or illnesses as well as depression or general cognitive improvement.

### BRIEF SUMMARY OF THE INVENTION

The present inventors have now discovered that a specific extract of North American ginseng, extracts of GB and SJW and their bioactive component ginsenosides Rb1, Rg1, Rc, Rd, Re, ginkgolides A and B, flavonoids quercetin and rutin, hypericin, and hyperforin have antioxidant and antiproliferative properties. The specific ginseng extract, which the inventors call HT1001 containing about 15-50%, preferably about 25-40%, total ginsenosides (a.k.a. saponins), can be administered alone or in combination with GB and/or SJW as an antioxidant or antiproliferative agent.

### BRIEF DESCRIPTION OF THE DRAWINGS

FIG. 1—Effect of herbal preparations on Fe²⁺-catalyzed lipid peroxidation in SH—SY5Y neuroblastoma cells. Cells were seeded at 1×10⁶ cells/well and grown for 24 hours prior to exposure to herbal extracts in the presence or absence of 0.1 mM FeSO₄ for 48 hours. Cell membranes were then harvested and assayed for lipid peroxides. The presence of lipid peroxides is indicated by formation of the colored MDA product and results are expressed relative to the control group. Herbal preparations are significantly different from control group with * P<0.05 using raw absorbance values.

FIG. 2—Prevention of Fe²⁺ interaction with ferrozine by herbal extracts. Various herbal extracts (FIG. 2b) and the potent Fe²⁺ chelator, DFO (FIG. 2a), were incubated in a cell-free chemical assay system at the indicated concentrations with 0.5 mM FeSO₄ solution followed by incubation with the chromogen, ferrozine (1 mM, final concentration). Results shown are absorbance at 562 nm of the test samples expressed relative to the appropriate blank groups.

FIG. 3—Prevention of Fe²⁺ interaction with ferrozine by pure chemical components of several herbal extracts. The pure chemicals quercetin, rutin, ginkgolide A, and ginkgolide B (FIG. 3a), ginsenosides Rb1, Rg1, Rc, Rd, Re (FIG. 3b), and hypericin and hyperforin (FIG. 3c) were incubated in a cell-free chemical assay system at the indicated concentrations with 0.5 mM FeSO₄ solution followed by incubation with the chromogen, ferrozine (1 mM, final concentration). Results shown are absorbance at 562 nm of the test samples expressed relative to the appropriate blank groups.

FIG. 4—Effect of herbal extracts on neuroblastoma cell proliferation in low and normal serum conditions. Various herbal extracts or vehicle alone (control) were incubated at 50 μg/ml with cultured SK—N—SH (FIG. 4a), SH—SY5Y (FIG. 4b) and N1E 115 (FIG. 4c) cells for 96 hours in either low (2%) or normal (10%) FBS. The effect of the Fe²⁺ chelator, DFO, on cell number was also investigated for both N1E 115 (solid symbols) and SH—SY5Y (unshaded symbols) cells (FIG. 4d). The number of viable cells was determined using the reagent WST-1. Significant inhibition of cell number is taken at *P<0.05 or **P<0.001 and significant stimulation of cell number is taken at # P<0.05 versus the same serum condition control group.

FIG. 5—Effect of ginkgolides, flavonoids, ginsenosides, and hyperforin on neuroblastoma cell proliferation. Ginkgolides A (GA) and B (GB) and the flavonoids rutin and quercetin (Q) (FIGS. 5a, b), or hyperforin or the ginsenosides Rb1, Rg1, Rc, Rd, and Re (FIGS. 5c, d), or vehicle alone (control) were incubated at the indicated concentrations with cultured N1E 115 (FIGS. 5a, c), SH—SY5Y (FIGS. 5b, d) cells for 96 hours. The number of viable cells was determined using the reagent WST-1. Significance is taken at *P<0.05, **P<0.001, ***P<0.0001 versus the control group.

FIG. 6—Dose-dependent effect of herbal extracts on N1E 115 neurite outgrowth. CVT-E033, HT1001, and GB were incubated at the indicated concentrations with cultured N1E 115 neuroblastoma cells for 7 days and neurite extension scores were determined. Significance is taken at *P<0.05 or **P<0.01 versus the control group.

### DESCRIPTION OF THE PREFERRED EMBODIMENTS

The present inventors have found that a proprietary extract of North American ginseng (HT1001) alone or in combination with GB and/or SJW has antioxidant and anti-cancer effects. The chemical constituents ginkgolides, flavonoids, ginsenosides, hypericin, and hyperforin, were also examined and found to have antioxidant activity. Antioxidant activity was found for all of the tested herbal extracts and their combinations through significant inhibition of Fe²⁺-catalyzed lipid peroxidation, the ability to chelate Fe²⁺ in a cell-free chemical assay system, and in the case of GB and CVT-E033 (North American ginseng plus GB), free radical scavenging. Furthermore, consistent with the behavior of Fe²⁺-chelators, anti-proliferative and/or pro-differentiative effects of all herbal extracts were demonstrated in several neuroblastoma models.

Antioxidant activity was exhibited with all herbal extracts tested and several of their chemical constituents. CVT-E033 (North American ginseng plus GB) and CVT-E036 (North American ginseng, GB, and SJW), were shown to reduce Fe²⁺-catalyzed lipid peroxidation in the human neuroblastoma cell line, SH—SY5Y. This may be attributable to contributions from HT1001, GB, and SJW which all independently inhibited Fe 2+-catalyzed lipid peroxidation. Extracts of GB (Ramassamy C, Girbe F, Christen Y, Costentin J. Ginkgo biloba extract EGb 761 or trolox C prevent the ascorbic acid/Fe²⁺ induced decrease in synaptosomal membrane fluidity. Free Radic Res Commun 1993; 19(5):341-50.), North American ginseng (Kitts D D, Wijewickreme A N, Hu C. Antioxidant properties of a North American ginseng extract. Mol Cell Biochem 2000 February; 203(1-2): 1-10), and SJW (Zou Y, Lu Y, Wei D. Antioxidant activity of a flavonoid-rich extract of Hypericum perforatum L. in vitro. J Agric Food Chem 2004 September 11; 52(16):5032-9) and flavonoid constituents (Chen Y T, Zheng R L, Jia Z J, Ju Y. Flavonoids as superoxide scavengers and antioxidants. Free Radic Biol Med 1990; 9(1):19-21) have previously been shown to inhibit lipid or liposome peroxidation. Fe²⁺-catalyzed lipid peroxidation may be directly antagonized through either free radical scavenging or Fe²⁺ chelation. CVT-E033 exhibited modest free radical scavenging activity; this was likely due to the GB component extract which demonstrated significant free radical scavenging, rather than the ginseng extract HT1100 which had no detectable activity. This is consistent with other reports of free radical scavenging by GB extracts (Ramassamy C, Naudin B, Christen Y, Clostre F, Costentin J. Prevention by Ginkgo biloba extract (EGb 761) and trolox C of the decrease in synaptosomal dopamine or serotonin uptake following incubation. Biochem Pharmacol 1992; 44(12):2395-401). In addition, all extracts exhibited Fe²⁺ chelation in the chemical model to a similar extent. Examination of some of the pure chemical components of these extracts revealed that quercetin (as reported previously in Sestili P, Guidarelli A, Dacha M, Cantoni O. Quercetin prevents DNA single strand breakage and cytotoxicity caused by tert-butylhydroperoxide: free radical scavenging versus iron chelating mechanism. Free Radic Biol Med 1998 August 15; 25(2):196-200), hypericin, and the 20-(s)-protopanaxadiol ginsenosides Rb1, Rc, and Rd, but notably not the 20-(s)-protopanaxatriol ginsenosides Rg1 and Re, displayed Fe²⁺ chelation activity indicating a unique structure-activity relationship. The data shown in the following examples suggest that CVT-E033, GB, HT1001, SJW, and CVT-E036 have distinct chemical components which may function as Fe²⁺ chelators. However, when each component was examined at a concentration which reflected their content in the herbal extracts, very little activity was shown suggesting that each component of an extract contributed synergistically or provided a summated effect in binding Fe²⁺.

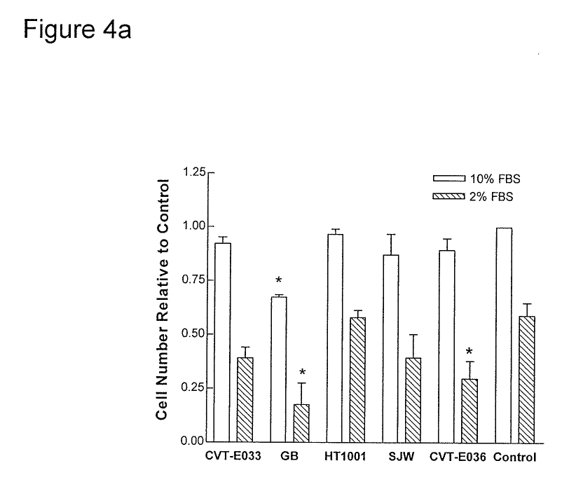

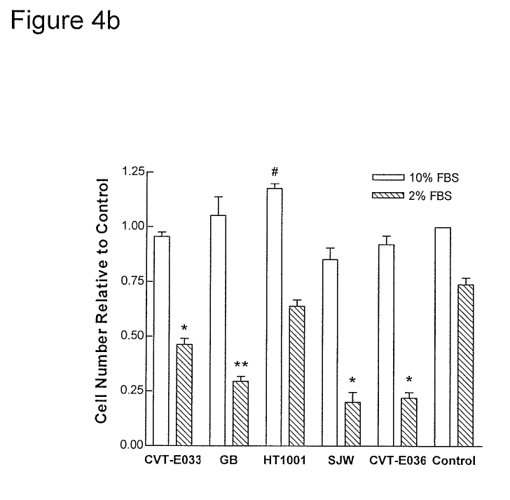

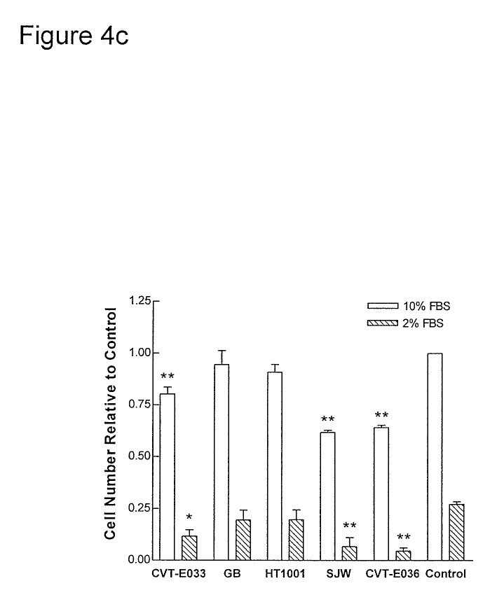

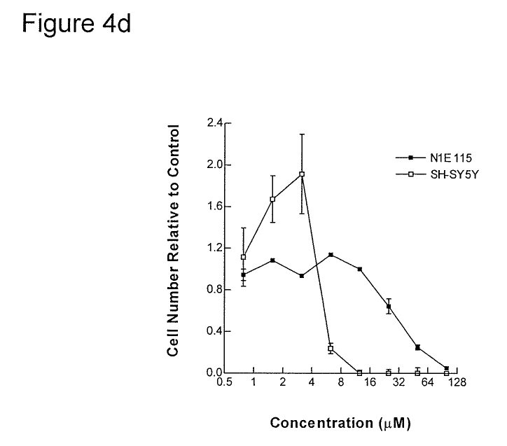

Anti-proliferative and/or pro-differentiative effects were observed for all extracts in various neuroblastoma cell lines. This is not surprising in light of the finding that all herbal preparations behaved as Fe²⁺ chelators, substances which have been shown to possess anticancer and anti-proliferative properties in neuroblastoma (Donfrancesco A, Deb G, De S L, Cozza R, Castellano A. Role of deferoxamine in tumor therapy. Acta Haematol 1996; 95(1):66-9). In the neuroblastoma culture models used in this study, the balance between Fe²⁺-chelation-related neuroprotective (i.e. pro-survival) activity (tested in the serum deprivation model) and anti-proliferative/pro-differentiative activity appears to be shifted in the direction of the latter. Of all the chemical constituents examined, only quercetin and hyperforin demonstrated inhibition of neuroblastoma cell number. Hyperforin (Dona M, Dell'Aica I, Pezzato E, Sartor L, Calabrese F, Della B M, Donella-Deana A, Appendino G, Borsarini A, Caniato R, Garbisa S. Hyperforin inhibits cancer invasion and metastasis. Cancer Res 2004 October 1; 64(17):6225-32), GB, and some flavonoids have been shown to exert anti-proliferative/pro-apoptotic effects in other neoplastic models (Huang Y T, Hwang J J, Lee P P, Ke F C, Huang J H, Huang C J, Kandaswami C, Middleton E Jr, Lee M T. Effects of luteolin and quercetin, inhibitors of tyrosine kinase, on cell growth and metastasis-associated properties in A431 cells overexpressing epidermal growth factor receptor. Br J Pharmacol 1999 December; 128(5):999-1010). Some specific ginsenosides have also been shown to exert anti-proliferative and pro-apoptotic effects in various neoplastic models, including human leukemia (THP-1) cells (Popovich D G, Kitts D D. Structure-function relationship exists for ginsenosides in reducing cell proliferation and inducing apoptosis in the human leukemia (THP-1) cell line. Arch Biochem Biophys 2002 November 1; 406(1):1-8), although notably not those ginsenosides examined during the present study (Rb1, Rg1, Rc, Rd, and Re), consistent with the results presented here. This is also consistent with the finding that the North American ginseng extract HT1001 alone was also not associated with inhibition of neuroblastoma cell number. A synergistic effect was observed between HT1001 and GB. CVT-E033 and CVT-E036 both decrease cell numbers under low serum conditions (stress) as compared to individual ingredients. CVT-E033 also is able to do this under non-stress conditions. The present inventors have found that when components were analyzed at concentrations which reflect their proportions in the combination of extracts, no significant effects on cell number were observed (FIG. 4c) thus the components appear to act synergistically.

In addition to their anti-proliferative/pro-apoptotic role, Fe²⁺ chelators have also been shown to induce cellular differentiation (Tanaka T, Muto N, Itoh N, Dota A, Nishina Y, Inada A, Tanaka K. Induction of differentiation of embryonal carcinoma F9 cells by iron chelators. Res Commun Mol Pathol Pharmacol 1995 December; 90(2):211-20). In this study, the extent of neurite outgrowth was quantified to provide an indication of neuroblastoma differentiation. CVT-E033, GB and HT1001 were all found to induce significant neurite outgrowth to a similar extent in N1E 115 cells. GB extract was observed to induce cell death in a high proportion of cultured neuroblasts, but those which survived exhibited a very high degree of neurite extension. Since HT1001 did not inhibit neuroblastoma cell number and even slightly enhanced cell number in SH—SY5Y cells, North American ginseng extract may work by both enhancing cell survival through an unknown mechanism, and inducing differentiation. Several crude ginseng extracts, as well as the ginsenosides Rb1 and Rg1, have previously been shown to promote neurite outgrowth directly, or to potentiate the stimulatory effects of nerve growth factor (NGF) on neurite outgrowth in N1E 115 cells (Rudakewich M, Ba F, Benishin C G. Neurotrophic and neuroprotective actions of ginsenosides Rb(1) and Rg(1). Planta Med 2001 August; 67(6):533-7). Furthermore, the ginsenosides Rb1 and Rb3 and notoginsenosides R4, 6 and Fa7 have also exhibited neurite outgrowth-promoting capabilities in SK—N—SH cells (Zou K, Zhu S, Meselhy M R, Tohda C, Cai S, Komatsu K. Dammarane-type Saponins from Panax japonicus and their neurite outgrowth activity in SK—N—SH cells. J Nat Prod 2002 October; 65(9): 1288-92).

There have been numerous reports of both neurotropic and neuroprotective effects of North American ginseng, GB, and SJW extracts both in vitro and in vivo. While much of these effects may be attributed to specific effects on neurotransmitter systems, anti-oxidant activity may also be a substantial contributor. The present invention provides the first description of antioxidant, iron chelating, and anti-proliferative/pro-differentiative effects in neuroblastoma for five standardized herbal and combination extracts of North American ginseng, GB, and SJW. The present invention also provides the first evidence of Fe²⁺ chelation activity for ginsenosides (20-(s)-protopanaxadiol ginsenosides) and the elucidation of a unique structure-activity relationship, and the SJW extract constituent, hypericin. The Fe²⁺ chelation activities of the examined extracts and some of their pure chemical bioactive components may be at least in part responsible for the anti-proliferative and pro-differentiative effects observed in neuroblastoma malignant cell lines.

A preferred extraction process for HT1001 is described in U.S. Pat. No. 6,083,932. In this process 300 kg of dried ground ginseng powder is used as a starting material. If desired, the ginseng can be placed into a Fitz mill and milled to about 80 mesh. The ginseng is then subjected to an extraction process using ethanol. It is preferred that 85% ethanol be used, though modification is well within the ordinary skill of a worker in the art. A solid:solvent ratio of about 1:5 to 1:10 is suitable, however 1:8 is preferred. Extraction can proceed for about 1 to 5 hours, as necessary. The preferred extraction time is 3 hours. Extraction temperature can be in a range of from 80-105° C., but 90-95° C. is preferred. Stirring is recommended. The liquid and solid phases are preferably separated with a decanter centrifuge at a speed of about 4200 rpm using a 25 micron in-line cartridge filter in the output line.

If a number of extractions are done, the supernatants can be pooled. At any rate, the supernatant is subjected to a concentration step to recover the ethanol. A vacuum distillation process is preferred. The target solid content is about 10-12° (Brix). The temperature is preferably about 50° C., at 15″ Hg, at a feed rate of 220-225 kg/h. Water may have to be added to avoid thickening.

The concentrated extract can be freeze, oven, or drum dried but is preferably spray dried at a feed temperature of about 42-58° C. and a feed rate of about 20 kg/h. The inlet temperature is preferably about 150-175° C., and the outlet temperature is preferably about 70-90° C. Once dried, the extract may be milled, if desired, to eliminate any lumps that may be present. It is preferred to use a Fitz mill fitted with a 0.065″ screen. The extract is then preferably blended to produce a yield of about 20%.

Gingko biloba extract can be obtained from commercial sources such as Acta Pharmacol (Sunnyvale, Calif.). Standardized extracts of Gingko biloba leaf contain about 24% Gingko flavone glycosides and about 6% terpene lactones as discussed in Sloley B D, Urichuk L J, Morley P, Durkin J, Shan J J, Pang P K, Coutts R T. Identification of kaemferol as a monoamine oxidase inhibitor and potential Neuroprotectant in extracts of Ginkgo biloba leaves. (J Pharm Pharmacol 2000 May; 52(4):451-9).

Saint Johns Wort extract can be obtained from commercial sources and are generally standardized to 0.3% hypericin content as discussed in Sloley B D, Urichuk L J, Ling L, Gu L D, Coutts R T, Pang P K, Shan J J., Chemical and pharmacological evaluation of Hypericum perforatum extracts (Acta Pharmacol Sin 2000; 21(12):1145-52.).

The specific ginseng extract, HT1001, containing about 15-50%, preferably about 25-40%, total ginsenosides (a.k.a. saponins), can be administered alone or in combination with GB SJW, ginkgolides, flavonoids, ginsenosides, hypericin, and/or hyperforin as an antioxidant or antiproliferative agent.

The antioxidant or antiproliferative agents according to the present invention can be administered by any suitable route including oral, aerosol or other device for delivery to the lungs, nasal spray, intravenous, intramuscular, intraperitoneal, vaginal, rectal, and topical or transdermal. Excipients which can be used as a vehicle for the delivery of the antioxidant or antiproliferative agent will be apparent to those skilled in the art. For example, the antioxidant or antiproliferative could be in a dried form and be dissolved just prior to administration by IV injection. Preferably administration of HT1001 alone or in combination with BB, SJW and/or active components thereof is oral or intravenous. If oral administration is intended, HT1001 is preferably made into 100 mg capsules. CVT-E033, a combination of HT1001 and GB, is preferably made into 125 mg capsules. CVT-E036, a combination of HT1001 and GB, is preferably made into 325 mg capsules (SJW:HT1001:GB, 200 mg:100 mg: 25 mg).

Suitable carriers, diluents or excipients are known in the art for preparing pharmaceutical compositions. Such suitable diluents or excipients include, but are not limited to, water, salt solutions, alcohols, gum arabic, vegetable oils, polyethylene glycols, gelatin, lactose, amylose, magnesium stearate, talc, starches such as corn or potato starches, silicic acid, viscous paraffin, fatty acid monoglycerides and diglycerides, pentaerythritol fatty acid esters, hydroxymethylcellulose, polyvinyl pyrrolidone, etc. The pharmaceutical composition can be sterilized and, if desired, mixed with auxiliary agents, such as lubricants, preservatives, stabilizers, wetting agents, emulsifiers, salts for influencing osmotic pressure, buffers, coloring, flavoring and/or aromatic substances. If administered alone, it is preferred that HT1001 is administered in a dosage range of 10-1000 mg of active agent for a 75 kg individual per day. It is more preferred to use a dosage of 75-500 mg per day, and most preferred to use a dosage of 100-200 mg per day.

The phrase “effective amount” as used herein includes the administration of HT1001 alone, or in combination with GB, SJW, ginkgolides, flavonoids, ginsenosides, hypericin, and/or hyperforin. The normally-effective amount of HT1001 can be reduced if the HT1001 is co-administered another antioxidant or antiproliferative agent. Thus, the term “effective amount” is intended to cover the use of HT1001 in combination with other effective ingredients, whereby the combination of the ingredients is effective, and the dosage of each ingredient can be proportionally reduced. For example, if HT1001 is normally administered effectively at 100 mg per individual per day, and Gingko biloba is normally administered effectively at 100 mg per individual per day, a combination of the two ingredients may be used whereby each is administered at 50 mg per individual per day. Such a combination use can be cost effective for the consumer if the other active ingredient (besides HT1001) is expensive, as is the case with many pharmaceuticals.

The composition according to the present invention can be used to treat oxidative stress, iron overload, and/or to promote neurite outgrowth. Oxidative stress and iron overload result from many conditions and diseases including but not limited to cancer, Alzheimer's disease, tuberculosis, Parkinson's disease, sickle cell, Wilson, liver damage, beta-thalassemia, heart disease, multiple sclerosis, inflammatory bowel disease (Crohn's disease and ulcerative colitis), infection, neoplasia, cardiomyopathy, and/or arthropathy. Iron loading in specific tissues is associated with an increased risk for disease (Eugene D. Weinberg, Iron Loading and Disease Surveillance, Emerging Infectious Diseases, Vol. 5, No. 3, May-June 1999 346-352). Iron acts as a pro-oxidant, stimulating the damaging effects in the body of substances known as free radicals. Table 1 below shows diseases that are associated with iron overload in various tissues.

TABLE 1

| Tissue type | Disease |
|---|---|
| Alveolar macrophages | Pulmonary neoplasia and infection |
| Anterior pituitary | Gonadal and growth dysfunction |
| Aorta; carotid and | Atherosclerosis |
| coronary arteries | |
| Colorectal mucosa | Adenoma, carcinoma |
| Heart | Arrhythmia, cardiomyopathy |
| Infant intestine | Botulism, salmonellosis, sudden death |
| Joints | Arthropathy |
| Liver | Viral hepatitis, cirrhosis, carcinoma |
| Macrophages | Intracellular infections |
| Pancreas | Acinar and beta cell necrosis, carcinoma |
| Plasma and lymph | Extracellular infections |
| Skeletal system | Osteoporosis |
| Skin | Leprosy, melanoma |
| Soft tissue | Sarcoma |
| Substantia nigra | Parkinson's disease |

### EXAMPLES

### Materials

Ferrozine, desferroxamine (DFO), quercetin, rutin, ginkgolide A, and ginkgolide B were obtained from Sigma. All extracts (HT1001, GB, SJW, CVT-E033, and CVT-E036) as well as the chemical component ginsenosides (Rb1, Rg1, Rc, Rd, and Re) were prepared and purified by CV Technologies (Edmonton, Alberta, Canada). Chemical characterization of the North American ginseng extract HT1001 (REMEMBER-fX®) (Sloley B D, Pang P K, Huang B H, Ba F, Li F L, Benishin C G, Greenshaw A J, Shan J J. American ginseng extract reduces scopolamine-induced amnesia in a spatial learning task. J Psychiatry Neurosci 1999 November; 24(5):442-52), GB extract (Sloley B D, Urichuk L J, Morley P, Durkin J, Shan J J, Pang P K, Coutts R T. Identification of kaemferol as a monoamine oxidase inhibitor and potential Neuroprotectant in extracts of Ginkgo biloba leaves. J Pharm Pharmacol 2000 May; 52(4):451-9.), and SJW (Sloley B D, Urichuk L J, Ling L, Gu L D, Coutts R T, Pang P K, Shan J J., Chemical and pharmacological evaluation of Hypericum perforatum extracts. Acta Pharmacol Sin 2000; 21(12):1145-52.) has been previously described. CVT-E033 (AD-fX®, 4:1 HT1001:GB extract (w/w)) and CVT-E036 (MENTA-fX®, 8:4:1 standardized extract of SJW: HT1001:GB extract (w/w)) are proprietary combination products standardized through ChemBioPrint™ and commercially available from CV Technologies. WST-1 cell proliferation reagent was purchased from F. Hoffmann-La Roche Ltd. (Postfach, Switzerland). Cell culture reagents were purchased from Gibco Life Technologies Canada (Burlington, ON, Canada). All other chemical reagents were purchased from either Sigma-Aldrich Chemical Co. (Oakville, Ontario, Canada) or Fisher Chemical Co. (Edmonton, Alberta, Canada). All samples, except for the water-soluble ginsenosides, were first dissolved in DMSO, followed by dilution in water for experiments.

### Lipid Peroxidation Assay

The ability of herbal extracts to inhibit lipid peroxidation was assessed using the thiobarbituric acid assay developed by Ohkawa et al (Ohkawa H, Ohishi N, Yagi K. Assay for lipid peroxides in animal tissues by thiobarbituric acid reaction. Anal Biochem 1979 July; 95(2):351-8) and the catecholaminergic neuroblastoma cell line SH—SY5Y (a generous gift from Dr. Peter Yu, University of Saskatchewan). Cells were maintained in Dulbecco's modified Eagle's medium (DMEM) supplemented with 10% fetal bovine serum (FBS) (v/v) and 1% antibiotic mixture (penicillin 50 μg/ml, streptomycin 50 μg/ml, 100 μg/ml; PSN) until approximately 90-100% confluency. They were then seeded in 6-well Nunclon tissue culture plates at 1×10⁶ cells/well and grown for 24 hours prior to exposure to herbal extracts in the presence or absence of 0.1 mM ferrous sulfate (FeSO₄) for 48 hours. Cells were then harvested and placed in disposable glass culture tubes, centrifuged, and the pellet resuspended in 0.4 ml 1.15% potassium chloride and homogenized with a sonic dismembrator. A 300 μl aliquot was then assayed for lipid peroxides using the method of Ohkawa et al with tetramethoxypropane as a reference standard. The remainder of material was assayed for protein content using the method of Lowry.

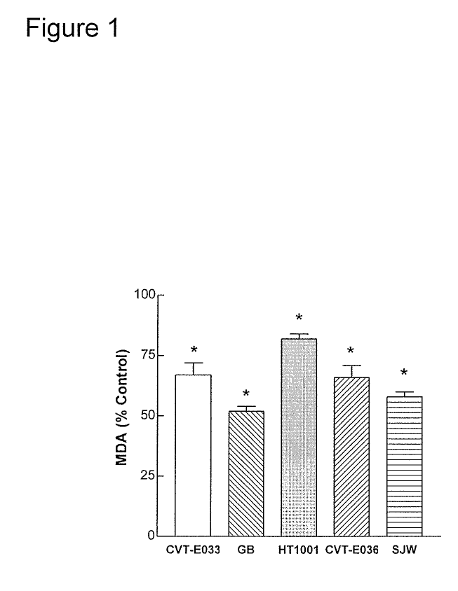

The effect of herbal extracts on lipid peroxidation in the presence and absence of 0.1 mM Fe²⁺ is shown in FIG. 1. All samples significantly (P<0.05) inhibited lipid peroxidation in the presence of Fe²⁺ only, but had no effect in the absence of Fe²⁺ (data not shown). The order of potency was GB>SJW>CVT−E033=CVT−E036>HT1001.

### Free Radical Scavenging

Hydroxyl free radical scavenging ability of herbal extracts and pure chemical components was assessed by a modification of the dynamic method developed by Amao et al (Arnao M B, Cano A, Hernandez-Ruiz J, Garcia-Canovas F, Acosta M. Inhibition by L-ascorbic acid and other antioxidants of the 2,2′-azino-bis(3-ethylbenzthiazoline-6-sulfonic acid) oxidation catalyzed by peroxidase: a new approach for determining total antioxidant status of foods. Anal Biochem 1996 June 1; 236(2):255-61). Briefly, herbal extracts were mixed with hydrogen peroxide and 2,2′-azino-bis(3-ethylbenzthiazoline-6-sulfonic acid) (ABTS) in a 50 mM Tris-HCl buffer (pH 7.2) and the reaction was initiated with the addition of 2 nM horseradish peroxidase. Hydroxyl radical scavenging activity of the sample was inferred by the ability to prevent formation of the colored ABTS radical (absorbance at 405 nm). Free radical scavenging activity for each sample was compared to a standard curve generated from ascorbic acid.

The free radical scavenging ability of the extracts GB, HT1001, and CVT-E033 is shown below in Table 2. Hydroxyl radical scavenging activity was inferred from the ability to prevent peroxidase-catalyzed formation of the ABTS radical. CVT-E033, and to a greater extent GB, displayed significant hydroxyl radical scavenging activity, whereas HT1001 had no detectable activity.

TABLE 2

| | ascorbic acid equivalent |
|---|---|
| Substance | by weight (%) |
| CVT-E033 | 0.183 ± 0.004 |
| HT1001 | n.d. |
| GB | 1.18 ± 0.021 |

Table 2. Free radical scavenging by North American ginseng extract HT1001, GB extract, and the combination CVT-E033 (HT1001 plus GB). Herbal extracts were incubated with hydrogen peroxide and ABTS and the reaction was initiated with the addition of horseradish peroxidase. Hydroxyl radical scavenging activity of the sample was inferred by the ability to prevent formation of the colored ABTS radical (absorbance at 405 nm). Free radical scavenging activity for each sample was compared to a standard curve generated from ascorbic acid and expressed as % ascorbic acid by weight.

### Fe²⁺ Chelation Assay

The ability of a herbal extract or pure chemical component to chelate Fe²⁺ in a cell-free assay system was assessed by the inhibition of formation of the colored Fe(ferrozine)₃²⁺ complex following incubation of FeSO₄ and the Fe²⁺-chelating chromogen, ferrozine. Ferrozine has previously been utilized to measure free Fe²⁺ release from ferritin. All herbal extracts and pure chemicals tested were prepared in 4% DMSO in water at 5 and 0.2 mg/ml respectively. A 50 μl aliquot was incubated with 50 μl of a 1 mM FeSO₄ solution in a 96-well microtiter plate (MTP) for 30 minute at room temperature. A 50 μl aliquot of the resulting solution was then transferred to a new 96-well MTP and combined with 50 μl of a 2 mM (saturating concentration) ferrozine solution. HT1001, GB, SJW, CVT-E033, and CVT-E036 were tested at 1.25 mg/ml and pure chemical components were tested at 50 μg/ml (final concentrations). Color formation was immediate and absorbance was read at 562 nm. Results were expressed relative to the blank group and activity of the samples was compared to the activity of the known Fe²⁺ chelator, DFO. DMSO was able to inhibit formation of the Fe(ferrozine)₃²⁺ complex therefore test extracts and chemical components were compared to an equivalent percentage DMSO blank.

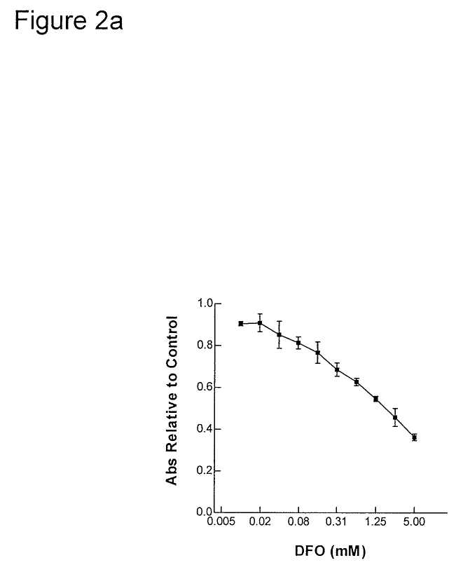

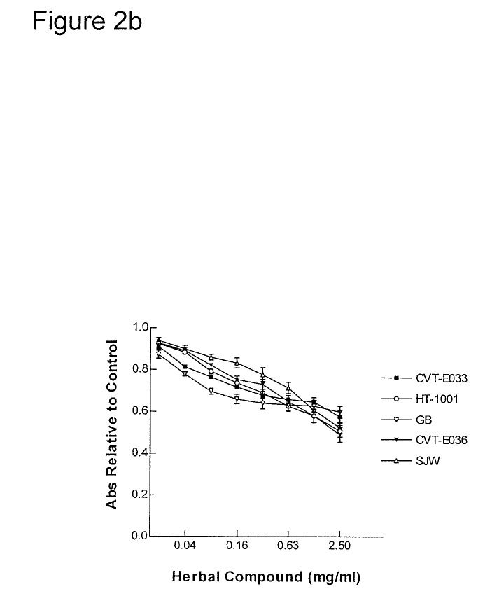

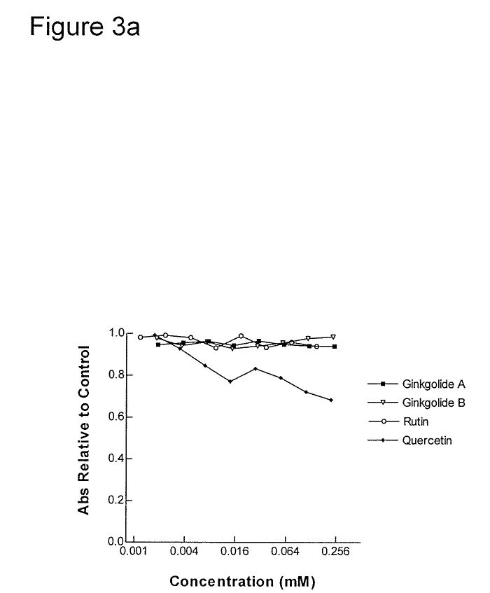

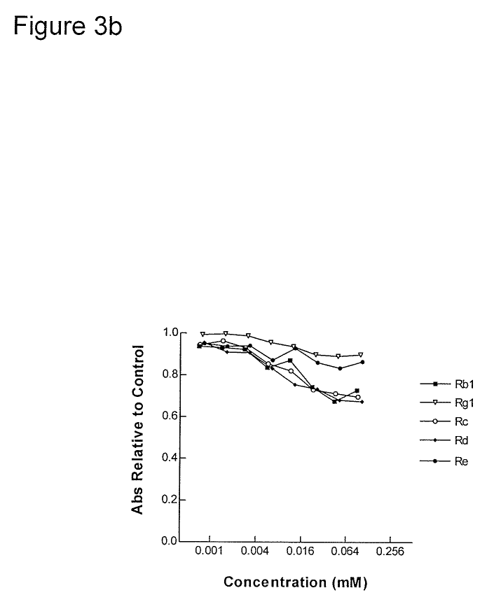

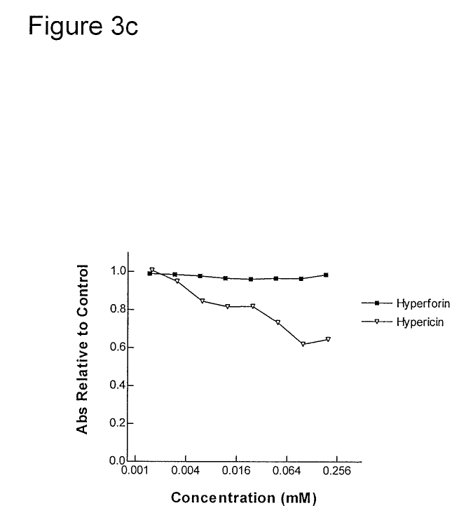

All herbal extracts and the pure chemical components ginkgolides A and B, the flavonoids quercetin and rutin, hyperforin, hypericin, and five ginsenosides, Rb1, Rg1, Rc, Rd, and Re were tested for the ability to inhibit Fe²⁺ binding to ferrozine in a cell-free chemical assay system. In order to obtain an adequate absorbance signal for the Fe(ferrozine)₃²⁺ complex, a non-physiological concentration of Fe²⁺ was chosen to react with ferrozine. This required high concentrations of the herbal extracts and components to potentially chelate Fe²⁺. Dose-dependent inhibitory effects on Fe(ferrozine)₃²⁺ complex formation of similar magnitudes were observed for CVT-E033, GB, HT1001, SJW, and CVT-E036 (FIG. 2b). The dose-response curve for the known Fe²⁺ chelator, DFO, is shown in FIG. 2a and indicates that DFO is able to prevent Fe(ferrozine)₃²⁺ complex formation in our assay system. The titration curves for ginkgolides A and B and the flavonoid rutin indicated no activity, whereas the flavonoid quercetin was able to inhibit Fe(ferrozine)₃²⁺ complex formation (FIG. 3a). The 20-(s)-protopanaxadiol ginsenosides Rb1, Rc, and Rd, but not the 20-(s)-protopanaxatriol ginsenosides Rg1 and Re, also demonstrated the ability to inhibit Fe²⁺ interaction with ferrozine (FIG. 3b). Analysis of the major known bioactive components of SJW showed that hypericin, but not hyperforin, was able to inhibit Fe(ferrozine)₃²⁺ complex formation (FIG. 3c). Taken together, these data suggest that CVT-E033, GB, HT1001, SJW, and CVT-E036 have distinct chemical components which may function as Fe²⁺ chelators. However, when each component was examined at a concentration which reflected their content in the herbal extracts, very little activity was shown suggesting that each component of an extract contributed synergistically or provided a summated effect in binding Fe²⁺.

### Neuroblastoma Cell Viability Assays

SH—SY5Y, N1E 115, and SK—N—SH (ATCC, Rockville, Md., USA) neuroblastoma cells were maintained by subculture in minimal essential medium (MEM) supplemented with 10% FBS (v/v), 1 mM sodium pyruvate, 2 mM L-glutamine and MEM non-essential amino acid mixture at 37° C. and under 5% CO₂ and 95% air. At approximately 80% confluency, cells were harvested and seeded in 96-well culture plates at 1×10⁴ cells/well containing herbal extracts or pure chemical components and various amounts of FBS. The final concentration of DMSO was not allowed to exceed 0.05%. The effect on cell number was evaluated after 96 hours by the addition of WST-1 cell proliferation reagent to each sample well according to manufacturer's specifications. Cell number was determined using the cell proliferation reagent WST-1 assay as previously described (Ba F, Pang P K, Benishin C G. The establishment of a reliable cytotoxic system with SK—N—SH neuroblastoma cell culture. J Neurosci Methods 2003 March 15; 123(1): 11-22). The accuracy of the WST-1 method as compared to the traditional trypan blue exclusion method of cell counting showed a strong correlation between the two techniques. Results were expressed relative to the control group.

The effects of all extracts were compared in three neuroblastoma cell lines at 50 μg/ml under both normal (10%) and low (2%) serum conditions. The results are shown in FIG. 4. The ginkgo-containing extracts GB, CVT-E033 and CVT-E036 were most often shown to exhibit suppression of cell number. Specifically, GB inhibited cell number in the human neuroblastoma lines SK—N—SH cells (low and normal serum) and SH—SY5Y (low serum). CVT-E033 in contrast significantly inhibited cell number in SH—SY5Y (low serum) and the murine neuroblastoma cell line N1E 115 (low and normal serum), and CVT-E036 suppressed cell number in all three cell lines in low serum conditions and additionally in N1E 115 in normal serum conditions. The extract of SJW also inhibited cell number in low serum conditions for SH—SY5Y and both conditions for N1E 115 cells. HT1001 had no inhibitory effects on cell number, and produced a small stimulation of cell number under high serum conditions in SH—SY5Y cells. The Fe²⁺ chelator DFO was also shown to suppress neuroblastoma cell number (FIG. 4d) in N1E 115 cells, but exert biphasic effects in SH—SY5Y cells.

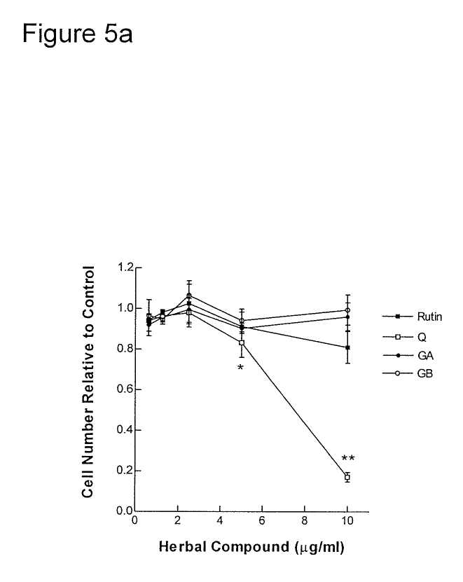

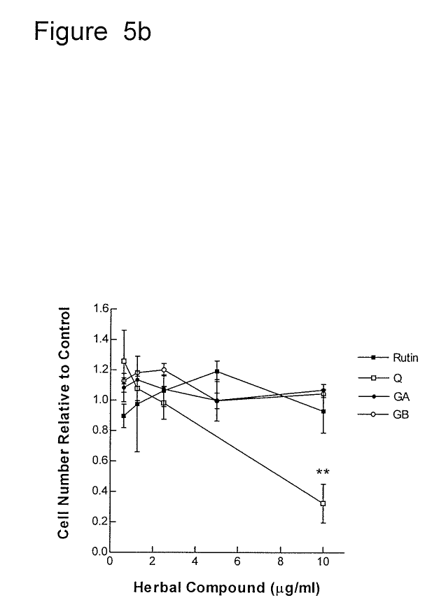

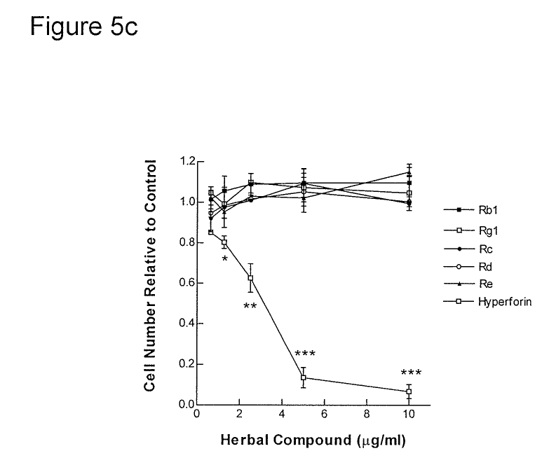

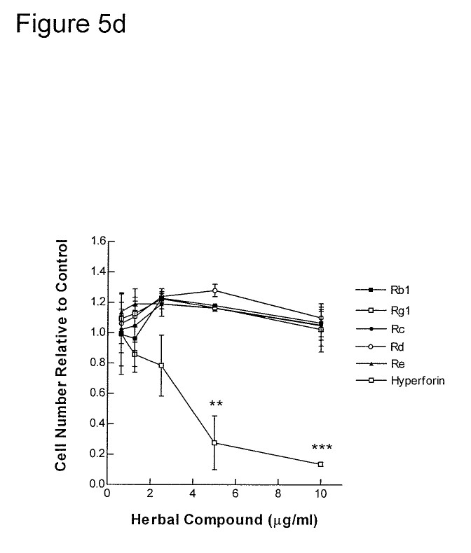

When individual components were analyzed at concentrations which reflected their proportions in the extracts, no significant effects on cell number were observed in any cell line. Because it was suspected that some portion of the compounds may have been lost during filtration, a dose-response curve was then constructed for all components in for both a murine model (N1E 115) and a human neuroblastoma model (SH—SY5Y). The dose-response curves for the ginkgolides (A and B) found in GB extract and CVT-E033 and CVT-E036 and flavonoids (quercetin and rutin) found additionally in SJW extract are shown in FIG. 5a (N1E 115 cells) and FIG. 5b (SH—SY5Y). Only quercetin demonstrated significant inhibition of cell number in both human SH—SY5Y and mouse N1E 115 cell lines with significance (P<0.05) first apparent at 5 μg/ml. The results for ginsenosides and hyperforin are shown in FIG. 5c (N1E 115 cells) and FIG. 5d (SH—SY5Y cells) and indicate that only hyperforin exhibited significant and dose-dependent suppression of cell number in both cell lines.

### Neurite Outgrowth

The murine neuroblastoma cell line N1E 115 was used to study the effects of the herbal extracts HT1001, GB, and CVT-E033 on neurite outgrowth. N1E 115 cells were seeded and maintained in 100 cm² tissue culture dishes at 37° C. in DMEM supplemented with 10% FBS and 1% PSN antibiotics (v/v). Cells were then mechanically dislodged and plated into 35 mm collagen-coated tissue culture dishes at a density of 2×10⁴ cells/dish. Neurite outgrowth was quantified for representative fields of cells after 7 or 14 days in culture with the herbal extracts. Two cell fields were photographed for each treatment dish and the extent of neurite extension was scored as follows: S1=cell became elongate or very little neurite outgrowth; S2=more than two small neurites extending from the cell body; S3=one or two neurites grew at least two times the cell body diameter; S4=more than two long neurites. The neurite index was calculates as:

Neurite index(In)=total neurite score(ΣS)/total cell number ΣN;

Where ΣS═S1*N+S2*N+S3*N+S4*N and N is the cell number of every cell field.

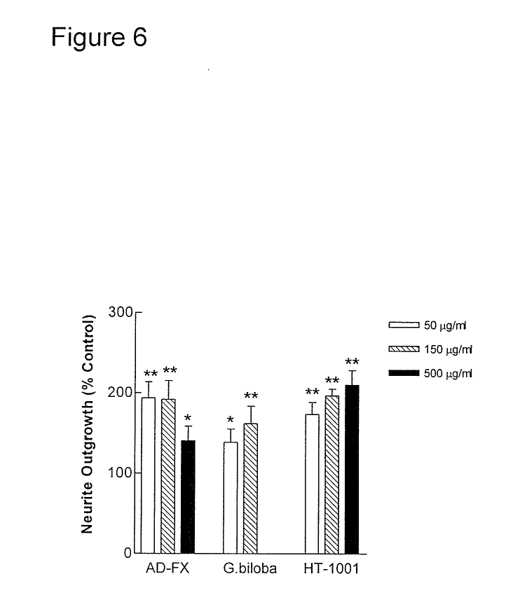

The effects of CVT-E033, HT1001, and GB on neurite extension are summarized in FIG. 6. All herbal extracts tested demonstrated the ability to induce neurite outgrowth from N1E 115 neuroblastoma cells to similar extent starting at 50 μg/ml. The effects of GB and HT1001 were dose-dependent. GB was observed to induce cell death (as supported with the above studies on cell number), but stimulate neurite outgrowth in those cells remaining.

### Statistical Analyses

Unless indicated otherwise, one-way analysis of variance (ANOVA) (Student-Newman-Keuls test) was used to determine significant differences, with the level of significance set at 0.05. All results are presented as mean ±standard error of the mean (SEM). Statistical analyses were performed using either GraphPad Prism or SigmaStat graphical and statistical software.

What is claimed is:

1-4. (canceled)

5. A method for treating oxidative stress, comprising administering, in an amount effective to reduce oxidative stress, a pharmaceutical composition comprising HT1001, an American ginseng extract that has a total ginsenoside content which is about 25-50% by weight, in combination with at least one agent selected from the group consisting of Ginkgo biloba, Saint John's Wort, ginkgolides, flavonoids, ginsenosides, hypericin, and hyperforin, to a patient in need of such treatment.

6. The method according to claim 5, wherein said oxidative stress is due to cancer, Alzheimer's disease, tuberculosis, Parkinson's disease, sickle cell, Wilson, liver damage, beta-thalassemia, heart disease, multiple sclerosis, inflammatory bowel disease (Crohn's disease and ulcerative colitis), infection, neoplasia, cardiomyopathy, or arthropathy.

7. A method for treating iron overload comprising administering, in an amount effective to reduce iron overload, a pharmaceutical composition comprising HT1001, an American ginseng extract that has a total ginsenoside content which is about 25-50% by weight, in combination with at least one agent selected from the group consisting of Ginkgo biloba, Saint John's Wort, ginkgolides, flavonoids, ginsenosides, hypericin, and hyperforin, to a patient in need of such treatment.

8. The method according to claim 7, wherein said iron overload is due to cancer, Alzheimer's disease, tuberculosis, Parkinson's disease, sickle cell, Wilson, liver damage, beta-thalassemia, heart disease, multiple sclerosis, inflammatory bowel disease (Crohn's disease and ulcerative colitis), infection, neoplasia, cardiomyopathy, or arthropathy.

9. A method for promoting neurite outgrowth comprising administering, in an amount effective to promote neurite outgrowth, a pharmaceutical composition comprising HT1001, an American ginseng extract that has a total ginsenoside content which is about 25-50% by weight, in combination with at least one agent selected from the group consisting of Ginkgo biloba, ginkgolides, flavonoids, and ginsenosides, to a patient in need of such treatment.

10. The method according to claim 9, wherein promoting neurite outgrowth is needed to treat Alzheimer's disease, Parkinson's disease, infection, and neuroblastoma.
